(19) United States
(12) Patent Application Publication
(10) Pub. No.: US 2016/0279162 A1
(43) Pub. Date: Sep. 29, 2016

(54) MICELLE-BASED DELIVERY OF DERMAL THERAPEUTIC MATERIALS

(71) Applicant: POLYREMEDY, INC., Castro Valley, CA (US)

(72) Inventor: David A. RICHARD, Shingle Springs, CA (US)

(21) Appl. No.: 15/037,312

(22) PCT Filed: Nov. 18, 2014
(86) PCT No.: PCT/US14/66236

§ 371 Date: May 17, 2016

Related U.S. Application Data
(60) Provisional application No. 61/905,395, filed on Nov. 18, 2013.
(60) Provisional application No. 61/939,791, filed on Feb. 14, 2014.

(51) Int. Cl. A61K 31/728; A61K 47/10; A61K 47/22; A61K 47/14; A61K 9/107; A61K 31/192; A61K 31/702; A61K 31/355; A61K 31/375; A61K 31/455; A61K 9/00; A61K 47/18

(52) U.S. Cl. CPC A61K 31/728; A61K 9/0014; A61K 47/10; A61K 47/22; A61K 47/14; A61K 47/18; A61K 31/192; A61K 31/702; A61K 31/355; A61K 31/375; A61K 31/455; A61K 9/1075

(57) ABSTRACT

Disclosed herein are compositions methods related to micelle-based delivery of dermal cosmetic and/or therapeutic materials. Micelles are formed by controlling proportions of hydrophilic and hydrophobic moieties, which then enables surfactants to facilitate formation of micelles and other cylindrical or vesicular nano-scale structures. These nano-scale structures are used to deliver therapeutic materials through the stratum corneum to underlying layers of skin.

### BACKGROUND

The present disclosure relates generally to therapeutic cosmetics. Specifically, the present disclosure relates to micelle-based delivery of dermal cosmetic or therapeutic materials.

The stratum corneum layer of the skin acts as a barrier to various foreign materials from entering underlying layers of the skin, such as the epidermis and the dermis. One aspect of barrier properties of the stratum corneum is that it not only prevents unwanted foreign materials from penetrating the skin, it also prevents materials intentionally applied to the skin from penetrating into the other layers of the skin. This inhibits the delivery of skin therapies to the underlying layers of the skin at which they could be of greater therapeutic value.

### SUMMARY

The present disclosure describes delivery of therapeutic materials through the stratum corneum to underlying layers of the skin without resorting to invasive application methods (e.g., iontophoresis, phonophoresis, and others). Rather, the delivery vehicle of the present disclosure is composed of micelles that include polyionic penetration enhancers and excipients. Micelles are formed by controlling proportions of hydrophilic and hydrophobic moieties, which then enables surfactants to facilitate formation of micelles and other cylindrical or vesicular nano-scale structures. These nano-scale structures are used to deliver therapeutic materials through the stratum corneum to underlying layers of skin. The therapeutic materials delivered include botanical and synthetic materials for the prophylactic or mitigation treatment of various skin aging problems such as a lack of skin firmness, wrinkles, and dry skin.

In one aspect, disclosed herein is a topical composition comprising: 5-25% poloxamer; 2-50% isopropyl alcohol; and 0-30% therapeutic or cosmetic component.

In some embodiments, the topical composition further comprises 1-50% of a penetration enhancer.

In some embodiments, the topical composition comprises about 4% or about 4.5% poloxamer.

In some embodiments, the poloxamer is poloxamer 188.

In some embodiments, the topical composition comprises about 4.7% laurocapram.

In some embodiments, the topical composition the penetration enhancer is octyl salicylate.

In some embodiments, the penetration enhancer is N,N-dialkyl-substituted amino acetate.

In some embodiments, the composition further comprises methanol, ethanol, other water-soluble alcohols, alkyl methyl sulfoxides, dimethyl acetamide, dimethyl formamide, pyrrolidones, propylene glycol, glycerol, silicone fluids, isopropyl palmitate, anionic surfactants, dioctyl sulphosuccinate, sodium lauryl sulphate, decodecylmethyl sulfoxide), bile salts, sodium ms taurocholate, sodium deoxycholate, sodium tauroglycocholate, propylene glycol-oleic acid, 1,4 butane diol-linoleic acid, urea, N,N-dimethyl-toluamide, calcium thioglycolate, anticholinergic agents, eucalyptol, di-o-methyl-beta cyclodextrin, or soyabean.

In some embodiments, the therapeutic or cosmetic component comprises 2-6% hyaluronic acid.

In some embodiments, the therapeutic or cosmetic component comprises about 1.6% hyaluronic acid.

In some embodiments, the therapeutic component is 1-3% 50 kDa hyaluronic acid and 1-3% 800 kDa hyaluronic acid.

In some embodiments, the topical composition further comprises 1-25% salicylic acid.

In some embodiments, the topical composition comprises about 4% salicylic acid.

In a further aspect, disclosed herein is a method of hydrating skin, the method comprising topically applying to skin any one of the compositions disclosed herein.

In a further aspect, disclosed herein is a method of treating rosacea or acne, the method comprising topically applying to skin any one of the compositions disclosed herein.

In a further aspect, disclosed herein is a method of delivering an active agent through the stratum corneum, the method comprising topically applying to skin any one of the compositions disclosed herein.

In a further aspect, disclosed herein is a topical composition comprising 95 wt. % hydration solution and 4.99 wt. % Argireline NP.

In a further aspect, disclosed herein is a topical composition comprising 98 wt. % hydration solution and 1.99 wt. % beta glucan.

In a further aspect, disclosed herein is a topical composition comprising 97 wt. % hydration solution and 2.99 wt. % tocopheryl acetate.

In a further aspect, disclosed herein is a topical composition comprising 97 wt. % hydration solution and 2.99 wt. % ascorbyl palmitate.

In a further aspect, disclosed herein is a topical composition comprising 95 wt. % hydration solution and 4.99 wt. % niacinimide.

In a further aspect, disclosed herein is a topical composition as in the above for use in the treatment of any of the conditions disclosed herein.

In a further aspect, disclosed herein is a method of preparing a micelle concentrate comprising the components of Table 1 by performing the steps disclosed herein.

In a further aspect, disclosed herein is a method of preparing a hydration comprising the components of Table 2 by performing the steps disclosed herein.

In a further aspect, disclosed herein is a method of preparing a bio-cellulose hydration fluid comprising the components of Table 3 by performing the steps disclosed herein.

In a further aspect, disclosed herein is a method of preparing a formulation for treating acne and/or rosacea comprising the components of Table 4 by performing the steps disclosed herein.

### BRIEF DESCRIPTION OF THE DRAWINGS

FIG. 1 is a plot showing transit of hyaluronic acid (”HA”) into the skin via tape strips collected from the surface of the skin specimen in contact with an HA mask for an incubation duration of one hour (“h”).

FIG. 2 is a plot showing cumulative (additive) transit of HA into the skin via tape strips collected from the surface of the skin specimen in contact with the HA mask for an incubation duration of 1 h.

FIG. 3 is a plot showing transit of a serum comprising both 50 kDa and 800 kDa HA into the skin via tape strips collected from the surface of the skin specimen in contact with the HA mask for an incubation duration of 1 h.

FIG. 4 is a plot showing cumulative (additive) transit of the combined 50 kDa/800 kDa HA into the skin via tape strips collected from the surface of the skin specimen in contact with the HA mask for an incubation duration of 1 h.

FIGS. 5A through 5G are diagrams showing transit of various embodiments of disclosed sera into the skin.

FIG. 6 is a plot showing transit of the HA into the skin grafts that were 500 μm in thickness.

FIG. 7A shows provider feedback data on patients' overall recovery after laser resurfacing. FIG. 7B shows provider feedback data on whether healing occurred more quickly as compared to standard of care protocols after laser resurfacing.

FIG. 8 shows provider feedback data on patients' level of face redness (A) and swelling (B) after laser resurfacing.

FIG. 9 shows provider feedback data on patients' level of pain (A) and swelling (B) after laser resurfacing.

FIG. 10 shows patient feedback on the patients' perception of healing (A) and face redness after laser resurfacing.

FIG. 11 shows patient feedback on the patients' perception of face swelling (A) and pain after laser resurfacing.

FIG. 12 shows patient feedback on the patients' perception of face itchiness (A) and (B) improvement of healing as compared to previous treatments after laser resurfacing.

FIG. 13 shows the effects of compositions on wrinkle reduction.

### DETAILED DESCRIPTION

### Overview

While many recent medical technologies exist for improving various skin conditions, including those associated with aging, many of these technologies involve side effects, adverse effects, and significant patient discomfort and/or recovery time. Unlike these existing technologies, the present disclosure describes delivery of therapeutic materials (including, but not limited to Hyaluronic acid (“HA”)) into the skin. The present disclosure describes formulations that pass through the stratum corneum to deliver therapeutic and cosmetic materials (hereinafter collectively “therapeutic materials”) to layers of skin under the stratum corneum.

Embodiments described herein use constituents that form micelles or vesicles that are capable of passing through the stratum corneum, which otherwise acts as an effective barrier against other conventional topically applied substances. Furthermore, hydrophilicity of the outer surface of micelles of the present disclosure can be tailored to pass through the stratum corneum and be absorbed in an underlying layer of skin resulting in much higher concentrations of the therapeutic under the stratum corneum than observed with conventional topically applied materials. Because of the absorption in an underlying layer of skin, therapeutic materials can be provided to underlying layers of skin more effectively and with higher absorption rates that those observed through conventional topically applied substances.

Furthermore, various therapeutic materials can be provided to one or more layers of skin under the stratum corneum that are targeted specifically.

### Micelle-Mediated Delivery Through the Stratum Corneum

Regardless of the material to be delivered, the stratum corneum acts as a barrier that prevents most materials from entering underlying layers of skin. However, it is these underlying layers that often benefit from exposure to therapeutic materials. To better deliver therapeutic materials and cellular enhancing constituents to these underlying layers, embodiments of the present disclosure include compositions that form micelles that carry these therapeutic materials.

The micelles are provided with other constituents in a topical serum to facilitate the passage of the micelles through the stratum corneum while minimizing, and at some times avoiding, damage or injury (e.g., exfoliation) to the stratum corneum. The topical serum includes four basic elements: 1) a block copolymer used to form a micelle around a core 2) a therapeutic material at the core of the micelle that is to be provided to skin layers underlying the stratum corneum 3) laurocapram (or other similar substance as described below) and 4) isopropyl alcohol (IPA). In such a system, a therapeutic material having a concentration in a serum from 2 wt. % to 5 wt. % (higher than typical drug loads which are ˜0.5 wt. %) can be topically applied with as much as 10 wt. % to 20 wt. % of the dosage delivered and absorbed by the targeted skin layer. This too is a higher percentage than the delivered dosages reported in the literature, which identify delivery rates through the stratum corneum of approximately 3 wt. %.

Substitutes (whether in whole or in part) to laurocapram include various solvents, surfactants, and other species. Examples of solvent substitutes to laurocapram include methanol, ethanol and other water-soluble alcohols, alkyl methyl sulfoxides (such as dimethyl sulfoxides, and alkyl homologs of methyl sulfoxide), dimethyl acetamide, dimethyl formamide, pyrrolidones (e.g., 2-pyrrolidone, n-methyl 2-pyrrolidone), and other solves such as propylene glycol, glycerol, silicone fluids, and isopropyl palmitate. Examples of surfactant substitutes include anionic surfactants (e.g., dioctyl sulphosuccinate, sodium lauryl sulphate, decodecylmethyl sulfoxide), and “bile salts” (e.g., sodium ms taurocholate, sodium deoxycholate, sodium tauroglycocholate). Other substitutes include propylene glycol-oleic acid, 1,4 butane diol-linoleic acid, urea, N,N-dimethyl-toluamide, calcium thioglycolate, anticholinergic agents, eucalyptol, di-o-methyl-beta cyclodextrin, and soyabean. A penetration enhancer can be selected in combination with the other components and for a particular application. For example, penetrations enhancers not generally regarded as safe would not typically be selected for over the counter cosmetic applications.

“Micelle” as described herein refers to a structure of any shape for convenience. Micelles include roughly spherical objects as well as roughly cylindrical objects (which are generally referred to as “vesicles”) or planar objects (which are generally referred to a “lamellae”). An individual micelle of the present disclosure carries molecules of a therapeutic material in its hydrophobic core.

In some embodiments, micelles (typically about 100 nm in diameter, although micelles can be reorganized into smaller sizes using ultrasonic agitation) are fabricated using non-ionic triblock co-polymers consisting of both hydrophilic and hydrophobic monomer units. The triblock copolymer molecules envelop a therapeutic material, thus carrying the therapeutic material through the stratum corneum for direct delivery to underlying skin layers while also protecting the therapeutic material from immediate hyaluraonidase attack.

In embodiments of the present disclosure, a poloxamer, a triblock copolymer of poly (ethylene oxide)-poly(propylene oxide)-poly(ethylene oxide) (PEO-PPO-PEO) is used. In one embodiment, the poloxamer used is poloxamer 188 which and has an average molecular weight of approximately 8400 g/mol. Poloxamer 188 is available from chemical supply companies such as Sigma Aldrich. Larger molecular weight polaxamers can also be used. While poloxamer has been used in biological contexts, it also has been noted that, contrary to results found in applications of embodiments of the present disclosure, “there should be little skin penetration and any penetration of the skin should be slow through the use of poloxamers.” Int J Toxicol. 2008; 27 Suppl 2:93-128. doi: 10.1080/10915810802244595. The Cosmetic Ingredient Review (CIR) Panel. Furthermore, concentrations of poloxamer used in the present disclosure are lower (approximately 5 wt. %) compared to poloxamer gels described in the literature, which describe uses of 15 wt. %. Poly(ethylene oxide)-Poly(propylene oxide)-Based Copolymers for Transdermal Drug Delivery: An Overview. Tropical Journal of Pharmaceutical Research, October 2012.

Testing showed that the rate of transit and absorption can be influenced by temperature. The release rate of the therapeutic material at the core of a micelle can be controlled by controlling whether the core molecule is more hydrophobic or less hydrophilic than the skin layer compared to the triblock co-polymer into which the molecule is incorporated. That is, if the therapeutic molecule at the core of the micelle has a greater affinity for the skin layer to which it is delivered, the therapeutic molecule will migrate from the micelle into the skin layer as a faster rate than if it has a lower affinity. Alternatively, a slower and more prolonged release rate of the molecule is possible when the hydrophobicity of the molecule is similar to that of the interior of the micelle.

The release rate of a molecule from a micelle core is, in part, a function of the physical and/or chemical properties of the micelle and the molecule at its core. The release rate is also a function of the in vivo conditions surrounding an absorbed micelle. In vivo factors include, but are not limited to, dilution, and the presence of proteins or other species that act to draw the molecule (or therapeutic material) out of the micelle.

### Example Compositional Ranges

While specific serum compositions are described below, the following section describes ranges of weight percentages of various components in example serums.

IPA is present in an example serum from 2% to 50% by weight, or alternatively from 5% to 25% or from 10% to 18%. Poloxamer 188 is present in one example serum from 2% to 50% by weight, or alternatively from 5% to 25% or from 10% to 15%. Hyaluronic Acid (50 Kda) is present in one of example serum from 0.5% to 4% by weight, or alternatively from 1% to 3% or from 1.5% to 2.5%. Hyaluronic Acid (800 Kda) is present in one example serum from 0.5% to 4% by weight, or alternatively from 1% to 3% or from 1.5% to 2.5%. Acetyl hexapeptide-3 is present in example serum from 1% to 50% by weight, or alternatively from 2% to 10% or from 3% to 8%. Caffeine is present in one example serum from 0.1% to 20% by weight, or alternatively from 0.2% to 10% or from 0.5% to 3.0%. Acetic acid is present in one example serum from 0.005% to 10% by weight, or alternatively from 0.010% to 8% or from 0.05% to 3%. Retinyl palmitate is present in one example serum from 0.05% to 15% by weight, or alternatively from 0.1% to 10% or from 0.2% to 5%. Tocopheryl acetate is present in one example serum from 1% to 25% by weight, or alternatively from 1.5% to 10% or from 2% to 8%. Ascorbyl palmitate is present in one example serum from 1% to 25% by weight, or alternatively from 1.5% to 10% or from 2% to 8%. Beta glucan is present in one example serum from 1% to 25% by weight, or alternatively from 0.5% to 10% or from 1% to 5%. Niacinimide is present in one example serum from 1% to 50% by weight, or alternatively from 2% to 10% or from 3% to 8%. Propylene glycol is present in one example serum from 1% to 50% by weight, or alternatively from 10% to 30% or from 15% to 25%. Azelaic acid is present in one example serum from 1% to 50% by weight, or alternatively from 5% to 30% or from 8% to 15%. Salicylic acid is present in one example serum from 1% to 25% by weight, or alternatively from 1.5% to 10% or from 2% to 8%. Laurie acid is present in one example serum from 1% to 25% by weight, or alternatively from 0.5% to 10% or from 1% to 5%. Glycerin is present in one example serum from 1% to 25% by weight, or alternatively from 0.5% to 10% or from 1% to 5%.

### Example Therapies Delivered Using Micelles

Therapeutic materials and cosmetics that can be delivered using embodiments of the present disclosure include, but are not limited to: botanical and synthetic materials for the prophylactic or mitigation treatment of various skin aging problems such as a lack of skin firmness, wrinkles, and dry skin; HA and nutrients to reduce wrinkles, improve tissue hydrodynamics, movement and proliferation of cells, while remaining substantially free of localized irritating or allergenic effects; at least one unsaturated fatty acid, salicylic acid, azelaic acid, niacinamide, lauric acid, propylene glycol, pluronic excipient, sodium chloride, hyaluronic acid, glycerin, sodium chloride, and water. The fatty acids include, but are not limited to oleic and elaidic acids. At least 1 beta hydroxy acid in an amount of about 8 weight %, at least 1 unsaturated fatty acid in an amount of about a weight of 10% weight, at least 1 vitamin that is a part of the vitamin B group in an amount of about 10%, at least 1 saturated fat in an amount of about 5% weight, at least 1 ionic compound in an amount of about 1% weight, a micelle concentrate of about an amount of 4% weight of hyaluronic acid, a secondary alcohol in an amount of about 10% by weight, a propylene glycol in an amount of about 25% by weight, glycerin in an amount of about 10% by weight, and a pluronic in an amount of about 8% by weight.

Embodiments of the present disclosure also include constituents that act to improve lipid penetration for use in transdermal delivery of therapeutic materials to skin layers underlying the stratum corneum. This can provide these underlying skin layers with nutrients and wrinkle reducing molecules using hyaluronic acid to improve tissue hydrodynamics, movement and proliferation of cells while remaining substantially free of irritants and allergens. In other words, the use of the lipid penetration enhancers enable even high molecular weight molecules like HA to pass through the stratum corneum.

The efficacious ingredients, including hyaluronic acid micelle precursors, beta hydroxy acid, fatty acids, and vitamins are best added above 40 degrees C. In some examples, the micelle precursors include, non-ionic surfactants, excipients, and penetration enhancers possess the least toxicity and skin irritation potential as opposed to anionic, cationic, and amphoteric.

In one embodiment of a therapeutic material composition, salicylic acid in an amount of 4 weight %, oleic acid in an amount of 5% weight, niacinamide in an amount of 10% weight, lauric acid in a 5% weight, sodium chloride in a 1% weight, a micelle concentrate with an amount of 2% weight of hyaluronic acid, isopropyl alcohol in an amount of 8% by weight, azelaic acid in an amount 10% by weight, propylene glycol in an amount of 20% by weight, glycerin in an amount of about 10% by weight, and a pluronic in an amount of about 5% by weight.

In some examples, proniosomes are added to micelle formulations in improve shelf life.

Embodiments of the present disclosure provide a topical cosmetic composition containing one or more of the following: monounsaturated fatty acid, isopropanol, non-ionic surfactants, propylene glycol, saturated dicarboxylic acid, 2-hydroxybenzoic acid, dodecanoic acid, nicotinic acid amide, hyaluronic acid, glycerin, polyhexamethylene biguanide, sodium chloride and water for improving or revitalizing the texture of skin or as a prophylactic against recurring skin irritation or degradation. Regardless of the ingredients, compositions have a pH of from 4.0 to 5.8 thereby substantially reducing irritation while maintaining a stable solution at room temperature.

Exemplary embodiments of the disclosed composition comprise: fatty acids including, but are not limited to, oleic and elaidic acids. At least 1 beta hydroxy acid, at least 1 unsaturated fatty acid, at least 1 vitamin that is a part of the vitamin B group, at least 1 saturated fat, at least 1 ionic compound, a micelle concentrate of hyaluronic acid, a secondary alcohol, a propylene glycol, a glycerin, and a pluronic.

### Example Applications

Embodiments of the present disclosure can be applied to the treatment of acne and rosacea. Therapeutic materials can be provided to the layers of skin underlying the stratum corneum that decrease the release of free fatty acids and other bacterial by-products that can trigger the lymphocyte reaction that transforms a microcyst into a papule and then into a pustule. The decrease in the bacterial byproducts reduces hyperkeratinisation of the follicular ostium, thus limiting the development of new acneic lesions, particularly non-inflammatory lesions (comedones).

Embodiments can also be used to for the effective skin treatment of various skin conditions, including wrinkles, erythema, dry skin, Rosacea and Acne, can be achieved with compositions which include unsaturated fatty acid, salicylic acid, azelaic acid, niacinamide, lauric acid, propylene glycol, pluronic excipient, sodium chloride, hyaluronic acid, glycerin, and water.

### Infusion Masque

In one embodiment, host infusion masques can effectively deliver HA therapeutically (as well as certain additional active molecules such as Argirerline, Beta Glucan, Tocopheryl Acetate, Ascorbyl Palmitate and Niacinimide) into the skin without the use of a procedure that causes the skin to heal after treatment.

Embodiments of the present disclosure include a host infusion masque used to deliver and/or provide for diffusion thereapeutic substance to the skin. At a high level, infusion masques of the present disclosure include can provide a convenient and effective delivery medium to skin for the various therapeutic micelle solutions described above.

A masque substrate was manufactured from a bio-(bacterial) cellulose. Gluconacetobacter xylinus (=Acetobacter xylinum, ATCC 10245) was purchased from the American Type Culture Collection and grown in 10 g/l Backtopeptone (Difco), 10 g/l yeast extract (Fisher), 4mM KH₂PO₄ (Sigma) and 20 g/l D-glucose dissolved in deionized water (DI). The pH of the medium was adjusted to 5.1-5.2.

The BC of the present disclosure has a three-dimensional non-woven network of nanofibrils sharing the same chemical structure as plant cellulose. The fibrils are held together by inter- and intra-fibular hydrogen bonding. From this hydrogen bonding and the natural hydrophillicity of cellulose, the BC of the present disclosure can be used to form a masque that is a hydrogel with high strength and high water retention.

Specifically, BC of the present disclosure has a high aspect ratio with a diameter of 20-100 nm. As a result, the selected BC has a very high area per unit mass. Because of the high area/unit BC mass and hydrophillicity of cellulose, BC of the present disclosure also has a high water absorption/unit BC mass ratio. The BC is a gel containing 99% percent water by weight, mainly due to its amorphous structure. Once fabricated, the masque can be used as a HA delivery tool.

In one example, the HA precursor and its host serum of fatty acid esters, polypeptides, polysaccharides, anti-oxidants and polymers was combined to effect a serum that is hydrated into a bio-cellulose infusion masque (described above) during masque fabrication to create effectively HA infused fibers. These fibers are capable of either picking up greater quantities of exudates when in dry gel form, or donating greater quantities of fluids when in hydrated state, due to the inherent nature of hydrogels to have significantly high absorptive and hydrative capacities. Additionally, the masque could also act as a scaffold to facilitate migration and proliferation of cells in the wound and promote more rapid wound healing.

Furthermore, it is possible to control distribution in the skin depending on the size of the micelle and to deliver HA onto a target depending on the surface properties of the target. That is, the size of the micelle can be tailored to match a corresponding size of structures in an under-lying layer of skin.

The solutions described herein can be tailored to target different layers of skin and/or provide a therapeutic material to a particular depth of skin under the stratum corneum by changing solution conditions such as pH, delivery temperature or electrolyte concentration. This tuning facilitates the release of the therapeutic material at a particular layer and/or depth. Furthermore, surface functionalization of micelles using biomolecules (e.g., saccharides and peptides) or other reactive functional groups can also be used to target various layers and/or structures in the skin. These reactive groups can be attached to the hydrophilic co-polymer chain terminus, thus changing the micelle surface chemistry to improve the targeting of micelles in subcutaneous delivery.

The hyaluronic acid (HA) used was a poly-anionic polysaccharide; particularly a thiol-derivatized hyaluronic acid. The HA is functionalized for E-Beam sterilization by adding one or more stabilizing excipients to the HA, or, in this case, by adding the HA to a formulation of stabilizing excipients. In this case we utilized the existing functionalizing precursor polymers in our fiber constituent bio-absorbable chemicals. Within the Polyethylene Oxide (PEO) constituent formulation used in the electrospinning of the dressing fiber there is contained Ethylene Vinyl Alcohol (EVAL). The EVAL content acts as an excipient to scavenge the effects of the E-Beam energy effectively stabilizing the polymer composition against the harmful effects of the sterilizing radiation. The normal effects to the HA, such as reduction in molecular weight, decreased solubility in aqueous solution, and alerted gelation times are mitigated. Further, the PEO component of the fiber solution acts on the HA as a covalent cross-linker, shielding the HA from the radiation effects by offering a host for the HA to adhere to.

### EXAMPLES

### Example 1

### Materials

Biocellulose masques containing HA with MW 50 kDa and 800 kDa, Argirerline, Beta Glucan, Tocopheryl Acetate, Ascorbyl Palmitate and Niacinimide, fabricated by Polyremedy, Inc., (San Jose, Calif.) were the primary components in this study. Tissue-Tek® O.C.T Compound was ordered from Sakura Finetek USA, Inc. HPLC grade water and Acetonitrile chemicals were ordered from VWR International, Inc. All other chemicals used in this study, not specifically identified, were also ordered from VWR International, Inc. Human abdominal skin samples obtained from post abdominoplasty procedures were used in this study for the assessment of skin permeation.

### Example 2

### Synthesis

In embodiments, a micelle solution (referred to below as “micelle concentrate”) is formed that can be combined with other therapeutic materials and/or solutions. One formulation of the first solution appears below in Table 1.

TABLE 1

| Total Volume of Solution: | Wt. % | 1000 ML | |
|---|---|---|---|
| DI Water | 93.67% | 936.7 | ml (+/−5 ml) |
| Surfactant (poloxamer-188) | 4.68% | 46.8 | g (+/−0.1 g) |
| Glacial Acetic Acid | 0.09% | 0.9 | ml (+/−0.01 ml) |
| Hyaluronic Acid, 50 KDA | 1.56% | 15.6 | g (+/−0.1 g) |
| Totals: | 100% | 1000 | ML |

The solution was prepared as follows: Deionized (“DI”) water was placed in a beaker, heated to 40° C. (−0° C./+5° C.), and stirred at between 100 RPM and 850 RPM for between 10 minutes and 20 minutes. The appropriate amount of poloxamer corresponding to the percentage in Table 1 was added slowly to the DI water while continuing to stir the heated DI water for at least one hour. Once the poloxamer was completely solvated in the water, the appropriate amount of glacial acetic acid corresponding to the percentage in Table 1 was added, while continuing to stir the heated solution. The mixture was stirred for at least 5 minutes. Then the appropriate amount of HA corresponding to the percentage in Table 1 was added to the mixture, while continuing to stir the heated solution for at least two hours. The pH of the solution is preferably between 4.0 and 5.0.

A similar solution was prepared using the same ingredients, weight percentages, and preparation method except that 800 kDA HA was used, instead of the 50 kDA HA shown in Table 1.

An enhanced hydration solution was prepared in another embodiment. One formulation of the enhanced hydration solution appears below in Table 2.

TABLE 2

| Total Volume of Solution: | Wt. % | 1000 ML | | 4000 ML | |
|---|---|---|---|---|---|
| Sterile Water | 70% | 700 | ml (+/−5 ml) | 2800 | ml (+/−10 ml) |
| Glycerin | 5% | 50 | ml (+/−1 ml) | 200 | ml (+/−5 ml) |
| Surfactant (poloxamer-188) | 5% | 50 | g (+/−0.1 g) | 200 | g (+/−2 g) |
| 70% IPA | 15% | 150 | ml (+/−1 ml) | 600 | ml (+/−5 ml) |
| Laurocapram | 5% | 50 | ml (+/−1 ml) | 200 | ml (+/−5 ml) |
| Totals: | 100% | 1000 | ML | 4000 | ML |

The enhanced hydration solution was prepared as follows: The sterile water was placed in a beaker, heated to 35° C.-40° C. and stirred at between 400 RPM-800 RPM for a minimum of 5 minutes. The appropriate amount of glycerin was added to the heated, stirring sterile water corresponding to the percentage in Table 2. The solution was stirred for at least 15 minutes. The appropriate amount of poloxamer corresponding to the percentage in Table 2 was added slowly to the above solution while continuing to stir the heated solution for at least two hours. Then, the appropriate amount of isopropyl alcohol (“IPA”) corresponding to the percentage in Table 2 was added slowly to the solution while continuing to stir for at least 15 minutes. Then, the appropriate amount of Laurocapram corresponding to the percentage in Table 2 was added slowly to the solution while continuing to stir for at least one hour.

Using the above micelle concentrates and the enhanced hydration solution, a bio-cellulose hydration fluid was synthesized according to the formulation shown below in Table 3.

TABLE 3

| Total Volume of Solution: | Wt. % | 1,000 ML | | 4,000 ML | |
|---|---|---|---|---|---|
| Enhanced Hydration Solution | 80.2% | 802 | ml (+/−10 ml) | 3,208 | ml (+/−20 ml) |
| Micelle Concentrate w/800 KDA HA | 5% | 50 | ml (+/−1 ml) | 200 | ml (+/−5 ml) |
| Micelle Concentrate w/50 KDA HA | 5% | 50 | ml (+/−1 ml) | 200 | ml (+/−5 ml) |
| Aloe Vera Oil | 3% | 30 | ml (+/−5 ml) | 120 | ml (+/−5 ml) |
| PHMB | 0.3% | 3.0 | ml (+/−0.1 ml) | 12 | ml (+/−1 ml) |
| Argireline NP Solution, a Hexa-Peptide | 5% | 50 | ml (+/−5 ml) | 200 | ml (+/−5 ml) |
| Caffeine Powder | 1.5% | 15.0 | g (+/−0.5 g) | 60.0 | g (+/−0.5 g) |
| Sodium Carbonate Monohydrate, g | TBD | TBD | | TBD | |
| (PN: 21-51551) | | | | | |
| Totals: | 100% | 1,000 | ML | 4,000 | ML |

The bio-cellulose hydration solution was prepared as follows: The enhanced hydration fluid (described above in the context of Table 2) was placed in a beaker and heated to 40° C. (−0° C./+5° C.), and stirred at between 100 RPM and 800 RPM for about 5 minutes (or as long as is required until the temperature equilibrates at 40° C. (−0° C./+5° C.)). The appropriate amount of Micelle Concentrate using 800 kDA HA corresponding to the percentage in Table 3 was added to the heated, stirring enhanced hydration fluid. Then, the appropriate amount of Micelle Concentrate using 50 kDA HA corresponding to the percentage in Table 3 was added to the heated, stirring solution. The solution was stirred for at least 5 minutes. Then, the appropriate amount of Aloe Vera oil corresponding to the percentage in Table 3 was added to the above solution and stirred for at least five minutes. Then, the appropriate amount of PHMB (polyhexamethylene biguanide) corresponding to the percentage in Table 3 was added to the above solution while continuing to stir the solution for at least 5 minutes. Then, the appropriate amount of Argireline NP solution corresponding to the percentage in Table 3 was added to the solution while continuing to stir for at least five minutes. Then, the appropriate amount of caffeine powder corresponding to the percentage in Table 3 was added to the solution while continuing to stir for at least five minutes.

Finally, sodium carbonate monohydrate was added to the solution while continuing to stir for at least five minutes. The amount of sodium carbonate monohydrate added is a function of the amount needed to bring the pH of the foregoing solution from 4.0 to 5.0. The solution was then placed in a bath of water in an ultrasonic device at 21.1° C. (+/−2° C.) and sonicated for at least 30 minutes. 0.5% of lavender oil can optionally be added after the caffeine power while maintaining the stirring rate and temperature described above for this solution.

In another embodiment, a formulation to treat acne and/or rosacea was synthesized using the above enhanced hydration solution, as shown in Table 4.

TABLE 4

| Total Volume of Solution: | Wt. % | 1,000 ML | | 4,000 ML | |
|---|---|---|---|---|---|
| Enhanced Hydration Fluid | 49.9% | 499 | ml (+/−5 ml) | 1996 | ml (+/−1 ml) |
| Propylene Glycol | 23% | 230 | ml (+/−5 ml) | 920 | ml (+/−1 ml) |
| Azelaic Acid | 10% | 100 | g (+/−0.1 g) | 400 | g (+/−0.1 g) |
| Salicylic Acid | 4% | 40 | g (+/−0.1 g) | 160 | g (+/−0.2 g) |
| Lauric Acid | 2% | 20 | g (+/−0.1 g) | 80 | g (+/−0.1 g) |
| Niacinamide | 5% | 50 | g (+/−0.1 g) | 200 | g (+/−0.2 g) |
| Sodium Chloride (NaCl) | 0.1% | 1 | g (+/−0.01 g) | 4 | g (+/−0.1 g) |
| Surfactant (poloxamer - 188) | 2% | 20 | g (+/−0.1 g) | 80 | g (+/−0.1 g) |
| Glycerin | 2% | 20 | g (+/−0.1 g) | 80 | g (+/−0.1 g) |
| Caffeine | 1.5% | 15 | g (+/−0.1 g) | 60 | g (+/−0.2 g) |
| Lavender (optional) | 0.5% | 5 | ml (+/−0.5 ml) | 20 | ml (+/−1 ml) |
| % Totals | 100% | 1,000 | ML | 4,000 | ML |

The acne/rosacea solution was prepared as follows: The enhanced hydration fluid (described above in the context of Table 2) was placed in a beaker and heated to 60° C. (−0° C./+5° C.), and stirred at between 100 RPM and 600 RPM for about 15 minutes (or as long as is required until the temperature equilibrates at 60° C. (−0° C./+5° C.)). Then, an amount of propylene glycol corresponding to the percentage in Table 4 was added to the heated, stirring enhanced hydration fluid and was stirred for at least 5 minutes. Then, an amount of azelaic acid corresponding to the percentage in Table 4 was added to the heated, stirring solution and was stirred for at least 5 minutes. Then, an amount of salicylic acid corresponding to the percentage in Table 4 was added to the heated, stirring solution and was stirred for at least 5 minutes. Then, an amount of lauric acid corresponding to the percentage in Table 4 was added to the heated, stirring solution and was stirred for at least 5 minutes. Then, an amount of niacinamide corresponding to the percentage in Table 4 was added to the heated, stirring solution and was stirred for at least 5 minutes. Then, an amount of NaCl corresponding to the percentage in Table 4 was added to the heated, stirring solution and was stirred for at least 5 minutes. Then, an amount of poloxamer 188 corresponding to the percentage in Table 4 was added to the heated, stirring solution and was stirred for at least 30 minutes or until the solution become clear. Then, an amount of glycerin corresponding to the percentage in Table 4 was added to the heated, stirring solution and was stirred for at least 5 minutes. Then, an amount of caffeine powder corresponding to the percentage in Table 4 was added to the solution while continuing to stir for at least five minutes. Finally, 0.5% of lavender oil can optionally be added after the caffeine power while maintaining the stirring rate and temperature described above for this solution.

In studies of embodiments of the present disclosure, different concentrations (0.1, 2, and 5%) of the PEO-PPO-PEO triblock copolymer were prepared by weight percentage basis. After the nonsulfated glycosaminoglycan/polymer was mixed with different concentrations (0.1, 2.0, 5.0%) of polymer with nonsulfated glycosaminoglycan (hyaluronic acid) (5.5 mg/ml.) the amount of nonsulfated glycosaminoglycan encapsulated in the of micelle/vesicle construct was calculated by measuring the difference between the total amount of nonsulfated glycosaminoglycan added in the PEO-PPO-PEO triblock copolymer preparation and the amount of non-trapped hyaluronic acid remaining in the aqueous solution. After the complex was formed at ambient temperature for 2 hours, two separate peaks of the complexes nonsulfated glycosaminoglycan/polymer and non-trapped nonsulfated glycosaminoglycan were determined by HPLC analysis (TSK-GEL G5000 PWXL column 0.7 mL/min flow rate of water (pH of 4.5) mobile phase). Loading efficiency of nonsulfated glycosaminoglycan—non-trapped nonsulfated glycosaminoglycan/total amount of nonsulfated glycosaminoglycan was found to be 58±3%.

Additionally, after application of the afore mentioned complex to a Gluconacetobacter xylinus based bio-cellulose film host, the film host was applied as an in-vivo application for the skin permeation evaluation of micelles formed using a combination of HA of 50 kDA and 800 KDA (as described above in the context of Table 1). Cryosections of permeated skin were analyzed by laser confocal microscope to observe distribution of the micelle concentrations. The results showed that a combination (non-distinguishable between 50 and 800 KDa molecular weight hyaluronic acid) were observed in all sections of the stratum corneum, epidermis and dermis of the penetrated skin. This is evidence of the transit of the nano-scale micelles (˜40 nm in diameter) for both low and high molecular weight HA.

These nano-polymeric particles have several advantages for trans-stratum corneum and dermal deliveries, including, among other benefits, increased flux, sustained release, and enhanced bio-availability of formulated molecules having skin enhancing capabilities.

Alternatively, a mixture of surfactant and co-surfactant (microemulsions) can be used as they present different thermodynamic stability and enhanced penetration through stratum corneum lipid disorganization acting in participation with penetration enhancers. Penetration enhancers include laurocapram, or other lipid disruptors, excipients such as IPA, and acetic acid.

### Example 3

### Skin Preparation

Upon arrival of fresh tissue, the skin specimens were placed between gauze pads that were soaked with 10× PBS (with 0.2% sodium azide). The arrangement was then placed into a Ziploc bag and stored in a −80° C. freezer. One night prior to expected experimentation, the frozen skin specimens were removed from −80° C. freezer. On the dissection board, using a scalpel the specimens were dissected into desired size pieces. Using surgical scissor, the hypodermis layer was removed but retained full thickness of the dermis and epidermis. A thin layer of wet gauze (hydrated with 10× PBS with 0.2% sodium azide) was placed on a digital hot plate. The tissue samples were placed in the laboratory incubator (maintained at 32° C.) elevated the temperature to produce a steady state temperature of approximately 32° C. on the skin surface. The skin surface temperature was monitored using a remote IR thermometer.

### Example 4

### Application of Test Material

The moisture on the skin surface was gently wiped off with dry gauze and then cleaned with a surfactant (0.5% DPPC). The test material was placed on the skin surface with the stratum corneum upward. The Franz cell receptor chamber was filled (8 mL capacity) with the PBS solution (pH 7.4) with 0.2% sodium azide (w/v). Each specimen was then be placed over the receptor chamber (stratum corneum facing upward) making sure to cover the active area on the receptor. A magnetic stir bar was already being placed in the receptor cell ahead of time. The material under test was placed flush over the skin on the stratum corneum and positioned accurately. Each receptor cell was then capped off with a donor cell on top of the skin sample and tightened with a clamp. All cells (skin permeation systems) were then placed on top of a magnetic stir plate (rotation speed 550 rpm) in an incubator preset at 32° C. The digital timer was set for a countdown as ascertained by the objective of the study.

### Example 5

### Tape Stripping Method

When the test duration time (1 hour in all tests) expired, the skin samples were removed from the laboratory incubator. On the dissection board, the material under test was gently removed. The surface of the skin was then cleaned with a damp gauze followed by 99% IPA damp gauze. Each skin specimen was visually inspected to determine if any residual remained. Using tweezers, scotch tape was applied on the specimen with the sticky side on the stratum corneum and rapidly removed. Note: an untreated (no test material) control sample was also processed and analyzed in identical conditions as the test samples. A total of 11 such tape applications were applied, the first of which was discarded due to potential contamination of the skin surface. The remaining tape samples were placed in 1.5 mL microcentrifuge tubes and subsequently extracted by vortexing at high speed for 1 minute followed with centrifugation at 12,000 rpm for 10 minutes at 4° C. The supernatant solution was then drawn out of each tube/container, filtered and analyzed with an HPLC system for the amount of active ingredient retained in the skin specimen under test. Averages of all samples tested were taken and recorded.

### Example 6

### Skin Permeation Method

A 500 μm thick strip of skin was heated to 32° C. and then grossed into pieces of 2 cm×2 cm. Each piece was visually inspected for any defects, and any samples that appeared compromised were discarded. The receptor chamber was filled (8 mL capacity) with the PBS solution (pH 7.4) with 0.2% sodium azide (w/v). Each skin specimen was placed over the receptor chamber (epidermis facing upward) making sure to cover the active area on the receptor. A magnetic stir bar was already placed in the receptor cell ahead of time. Each receptor cell was capped off with the material under test followed with a donor cell on top of the skin sample and tightened with a clamp. All cells (skin permeation systems) were placed on top of a magnetic stir plate (rotation speed 550 rpm) in an incubator preset at 32° C. Aliquots (1 mL) were extracted and fresh solution was correspondingly replaced from each diffusion cell at intervals of 15 minutes, 30 minutes, 1 hour, 2 hours, 4 hours and 6 hours, 8 hours and 24 hours. The aliquots were filtered and analyzed using reversed phase HPLC.

After the 24-hour permeation test was complete, the skin graft under test was washed with 5% soap solution. Then a biopsy punch (6 mm) was excised from the area in direct contact with the material under test and weighed (the weight of each sample was used to determine the approximate thickness of the sample, both of which were used for normalizations to ideal weight given an ideal thickness of 500 μm. This biopsy punch was diced using a scalpel and then homogenized for about 60 seconds in 10 seconds pulses followed by centrifugation (4° C.) at 10,000 rpm for 10 minutes. The supernatant solution was then drawn out of each tube/container, filtered and analyzed using HPLC for the amount of actives retained in the skin specimen under test. Total uptake was calculated as the sum of the normalized cumulative permeation and the normalized retention in each sample. Averages of all samples tested were taken and recorded.

### Example 7

### Solution Sampling and HPLC Parameters

Volume Extracted (mL), Vₑ: volume of sample solution extracted from each diffusion cell at each time interval. Diffusion Cell Volume (mL), Vₒ: original volume of solution within each diffusion cell. Replacement Volume (mL) Vᵣ: volume returned to diffusion cell after each extraction, and has to be the same as Volume Extracted (Vᵣ=Vₑ), to make up a total volume which must be equal to the original Diffusion Cell Volume. Injection Volume (mL), Vᵢ; volume of sample solution injected for HPLC analysis. Dilution Factor, D: Magnitude of sample solution diluted before injection for HPLC analysis.

### Example 8

### Calibration Curve

For each Molecule-Under-Test, a calibration curve was created with the HPLC system prior to the experiments. A few samples of the active under test were prepared at known concentration (mg/mL) which was analyzed with the HPLC system for chromatographic absorbance (mAU). The absorbance peak for the molecule was identified by its retention time in the separation column compartment. A graph of a simple linear algebraic equation Y=mX was plotted, where Y=Absorbance (mAU) and X=Mass (μg). The slope, m was noted and used later to calculate the actual total mass of molecule permeated, X_{t,n}, through the skin samples during the experiments, given the absorbance measured by the HPLC system, where t=sampling time interval and n=index number of the skin permeation system. At each time interval, Vₑ−1 mL sample solution was extracted from each permeation cell, and Vᵣ=1 mL was returned to each permeation cell. An injection volume, Vᵢ, for HPLC analysis from each sample solution was determined, along with an optional dilution factor, D. During each analysis, the HPLC system returned an absorbance value, Y. The actual volume or mass of the Molecule-Under-Test was calculated using the calibration curve. The volume or mass of sample solutions, X_{1,n}, taken on the first time interval was calculated using:

$${X}_{1 , n} = \frac{Y - C}{m} \cdot \{ \frac{{V}_{o} D}{{V}_{i}} \}$$

The mass of sample solutions, X_{t>1,n}, taken on subsequent intervals was calculated using:

$${X}_{t > 1 , n} = \frac{Y - C}{m} \cdot \{ \frac{{V}_{o} D}{{V}_{i}} \} + \frac{{X}_{( t > 1 ) - 1 , n} {V}_{e}}{{V}_{o}}$$

The permeation of the molecule under test per cm² was calculated by knowing the effective area of the treated skin specimen in direct contact with the topical solution in the donor cell. The area of skin in contact with the topical solution was equivalent to the area of the opening slit on the donor cell.

Therefore, Permeation

$${P}_{t , n} = \frac{{X}_{t , n}}{A} mg \text{/} {cm}^{2}$$

Where A=area of skin in contact with molecule under test (1 cm diameter). The average of all the samples at each time point was taken and plotted with permeation of the molecule under test as a function of time. Based on the measurements and calculations, the samples evaluated at 24 hours provided a cumulative amount permeated past the skin graft under test. Since the graft thickness was known to vary, the permeation amount was normalized to a 500 μm skin graft thickness by using:

$${X}_{24 hours , n} = ( \frac{Y - C}{m} \cdot \{ \frac{{V}_{o} D}{{V}_{i}} \} + \frac{{X}_{( t > 1 ) - 1 , n} {V}_{e}}{{V}_{o}} ) \cdot ( \frac{{t}_{m}}{{t}_{i}} )$$

Where tₘ was the measured graft thickness and tᵢ was the ideal graft thickness which was 500 μm. Averages of the samples were taken in order to determine the mean and standard deviation for permeation. Permeation ratio and ratio standard deviation could then be calculated. At the end of the permeation testing period (24 hours) a 6 mm biopsy punch was taken from the center of the skin specimen that was in contact with the topical solution under test. The biopsy was weighed in grams. Once the biopsy was processed and the resultant aliquot absorbance value was obtained, the mass of molecule retained was calculated using:

$${Z}_{t , n} = \frac{Y - C}{m} \cdot \{ \frac{{V}_{c} D}{{V}_{i}} \}$$

Where V_{c}=volume of supernatant in centrifuge tube and t=24 hours. This quantity was normalized for the area of the skin in contact with the molecule under test and for differential mass (weight of sample) compared to the ideal mass for a 500 μm graft thickness. This was calculated by:

$${R}_{t , n} = ( \frac{Y - C}{m} \cdot \{ \frac{{V}_{c} D}{{V}_{i}} \} ) \cdot ( \frac{{A}_{N}}{{A}_{B}} ) \cdot ( \frac{{M}_{i}}{{M}_{m}} )$$

Where A_{N} =normalized area of skin in contact with molecule under test, A_{B}=biopsy area, Mᵢ=ideal mass for 500 μm graft thickness, Mₘ=mass measured on each biopsy. Averages of the samples are taken in order to determine the mean and standard deviation for retention. Retention ratio and ratio standard deviation were also calculated. Normalized uptake of the molecule under test was calculated by using:

U_{t,n}=X_{24 hours,n}+R_{24 hours,n} in mass of the molecule uptaken by the skin specimen in contact with the test solution. Uptake in mass per unit area was further calculated by

(U_{t,n}=X_{24 hours,n}+R_{24 hours,n})/A

Where A=area of skin in contact with molecule under test. Averages of the samples were taken in order to determine the mean and standard deviation for uptake enhancement. Uptake ratio and ratio standard deviation were then calculated.

### Example 9

### Histological Processing

Biopsy specimens were taken from 1 hour exposure tests and embedded in Optimal Cutting Fluid Temperature fluid for frozen sectioning. Samples were frozen sectioned at thickness 10 μm and collected on charged microscope slides. Images were taken using a light microscope with a preinstalled digital camera (Leica, Inc.) using light and cross polarized filtering. In addition to the two molecular weights being tested (50 and 800 kDa) there were also tests carried out for individual constituents delivered through the following serums:
Serum 1: 5% Argirerline
Serum 2: 2% Beta Glucan
Serum 3: 3% Tocopheryl Acetate
Serum 4: 3% Ascorbyl Palmitate
Serum 5: 5% Niacinimide

### Example 10

### Results & Discussion

The preponderance of studies was carried out with 800 kDa molecular weight hyaluronic acid (HA). This was done to have uniformity in gauging the changes in uptake through penetration enhancer and excipient modifications. Some defining studies were carried out to discern difference in performance in uptake of 50 kDa versus 800 kDa molecular weight HA. Further, a distinct transit evaluation of a combined content serum having both 50 kDa and 800 kDa HA was performed. Furthermore, histological testing was carried out on samples exposed to additional individual active ingredients, presented within the embodiment of the ensuing results. Note, quantitative measurements were only carried out using HA and not assessed for the other active ingredients.

### Example 11

### Tape Stripping Results

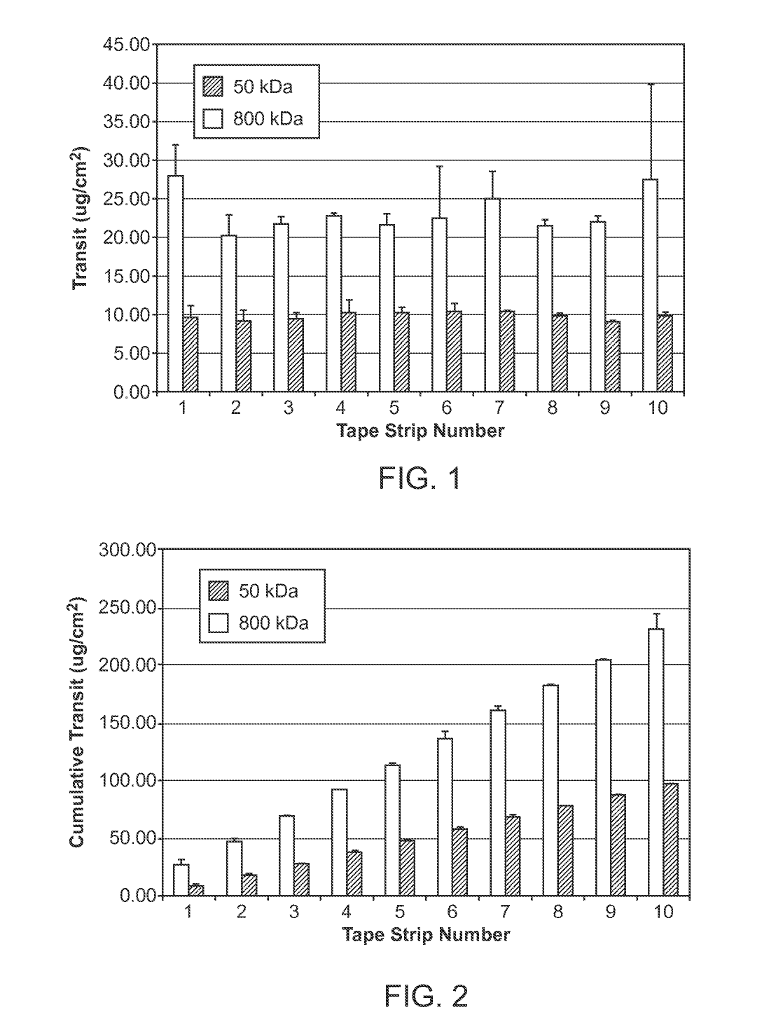

A common tissue donor was used with three independent site applications of the test material masks were tested on different areas of the tissue specimens through an incubation period of 1 h. Both, the 50 kDa and the 800 kDa masks showed transit into the skin specimen at this incubation point (FIG. 1). Also noted from the results was that the 50 kDa samples showed greater transit into the skin when compared with the 800 kDa samples. The cumulative transit into the skin progressively increased as a function of depth into the skin (FIG. 2). This plot is simply an additive representation of the data shown in FIG. 1.

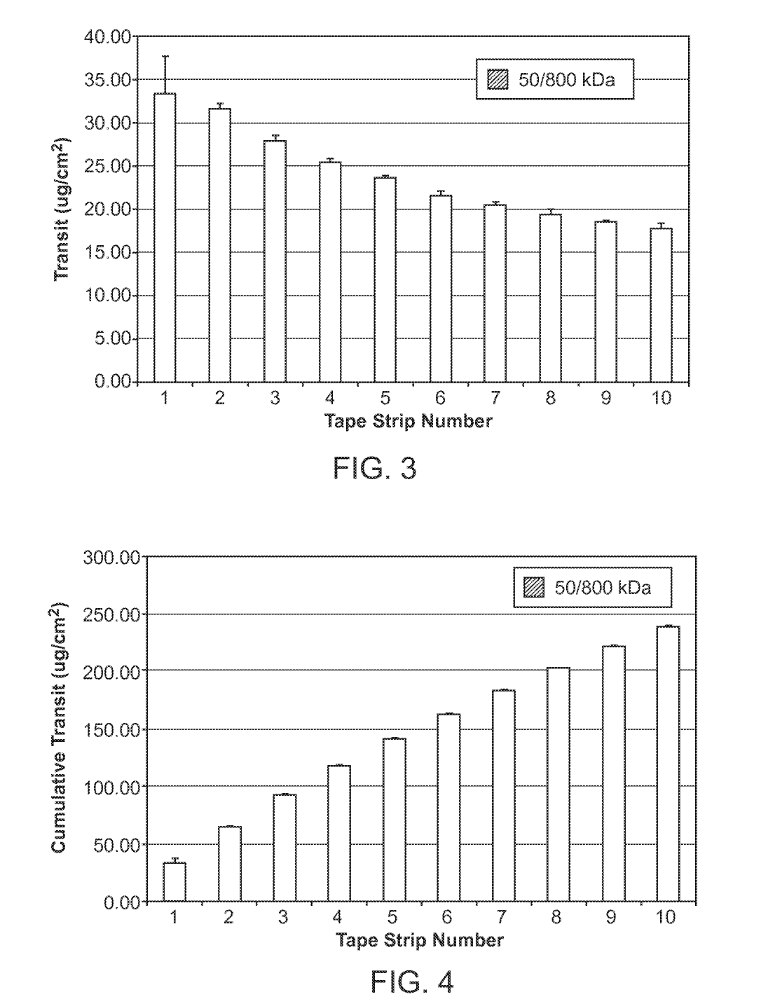

Also tested within the paradigm were mask samples that were loaded with both isotopes of HA, 50 kDa and 800 kDa, all within the same samples. The test protocols followed were identical to those where samples contained only one molecular weighted form of HA. The test results for individual transit and cumulative transit are shown in FIGS. 3 and 4 respectively. To be noted in these results was that despite the visible quantifiable transit of HA into the skin, the two isotopes were indistinguishable traces from one another.

FIG. 1 is a plot showing transit of the HA into the skin via tape strips collected from the surface of the skin specimen in contact with the HA mask for an incubation duration of 1 h. Note greater transit in the 50 kDa samples, while still significantly visible transit with the 800 kDa samples.

FIG. 2 is a plot showing cumulative (additive) transit of the HA into the skin via tape strips collected from the surface of the skin specimen in contact with the HA mask for an incubation duration of 1 h. Note greater transit in the 50 kDa samples, while still significantly visible transit with the 800 kDa samples. This plot is simply an additive representation of the data shown in FIG. 1.

FIG. 3 is a plot showing transit of the combined 50 kDa/800 kDa HA into the skin via tape strips collected from the surface of the skin specimen in contact with the HA mask for an incubation duration of 1 h.

FIG. 4 is a plot showing cumulative (additive) transit of the combined 50 kDa/800 kDa HA into the skin via tape strips collected from the surface of the skin specimen in contact with the HA mask for an incubation duration of 1 h. This plot is simply an additive representation of the data shown in FIG. 3.

### Example 12

### Histological Results

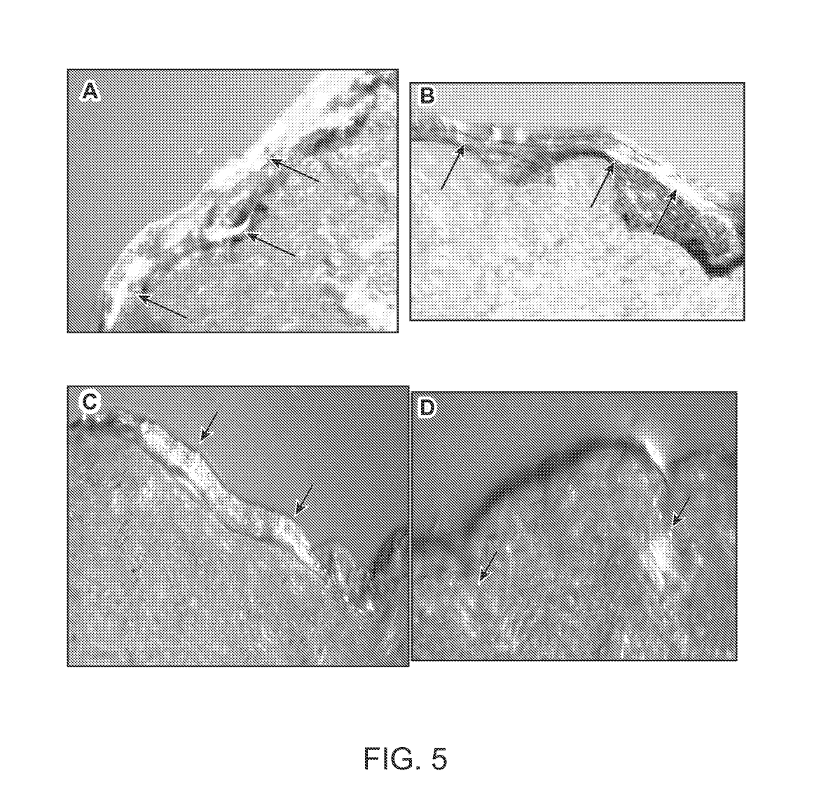

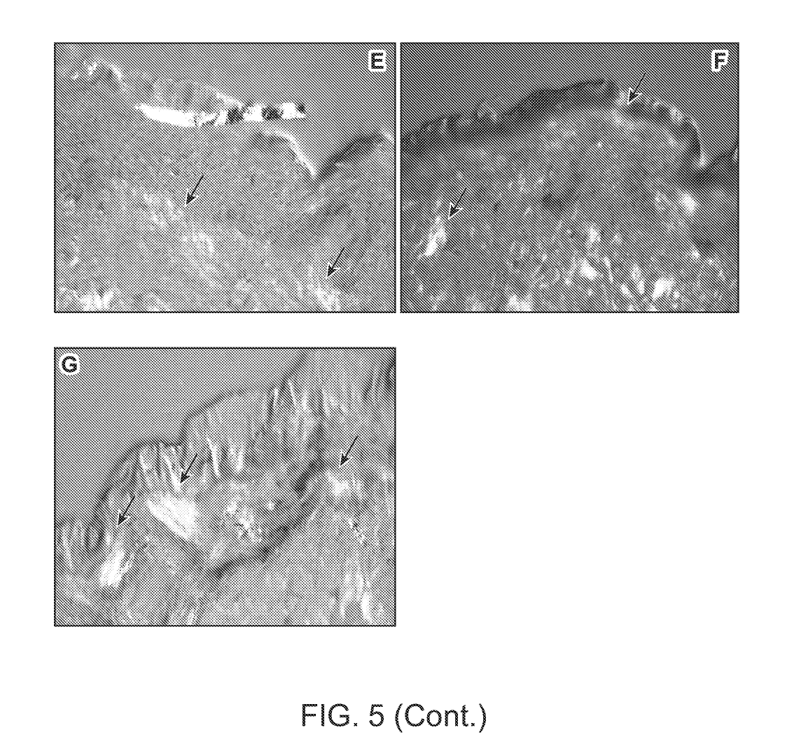

For histological examination and visual indication of transit, the micelles were loaded with trypan blue dye. Mask samples were tested in identical conditions as used in the tape stripping tests, i.e. biopsies were taken from skin specimens after 1 h incubation and frozen sectioned. Results showed visible transit of the dye into the skin as can be seen from FIG. 5.

FIGS. 5A-5G. Histological illustration showing transit of the micelle infused trypan blue dye into the skin specimen when using a) 50 kDa, b) 800 kDa HA, c) 5% Argirerline, d) 2% Beta Glucan, e) 3% Tocopheryl Acetate, f) 3% Ascorbyl Palmitate and g) 5% Niacinimide masks. The yellow indicators show the presence of the dye, microscopically. Both images are represented at 10× magnification.

The activity of the individual constituents to transit and take residence in the dermis is mainly due to the use of a specifically formulated amphiphilic block copolymer that allows for self-assembly into a three-dimensional spherical micelle structure or a nanorod-like micelle structure. This specific micelle structure is now shown to efficiently carry a variety of specific zeta charged cargos to skin cells.

Serum 1 (Argireline): Serum 1 is composed of 95 wt. % hydration solution (described above), 4.99 wt. % Argireline NP, and 0.01% trypan blue (mixed with the Argireline by ultrasonic agitation for 15 minutes at 70° F. and used as a dye to determine the interaction of the serum with skin). The Argireline is acetyl hexapeptide-3, sometimes referred to as hexapeptide-8 (sold as ARGIRELINE™). Acetyl hexapeptide-3 has sequence Ac-Glu-Glu-Met-Gln-Arg-Arg-NH₂ (SEQ ID NO:1). Acetyl hexapeptide-3 mimics the N-terminal end of the SNAP-25 protein that inhibits the soluble N-ethyl-maleimide-sensitive factor attachment protein receptor (SNARE) complex formation“. In FIG. 5C, it appears that the tagged hexapeptide has transited the stratum corneum and saturated the epidermis and infiltrated into the dermis.

Serum 2: Serum 2 is composed of 98 wt. % hydration solution (described above), 1.99 wt. % beta glucan, and 0.01 wt. % trypan blue (mixed with the beta glucan by ultrasonic agitation for 15 minutes at 70° F.). Beta-glucan (often from oats) is known as a skin moisturizer and has a history of healing minor wounds and burns. FIG. 5D confirms that the molecules are small enough to penetrate the stratum corneum, epidermis and reached the dermis.

Serum 3: Serum 3 is composed of 97 wt. % hydration solution (described above), 2.99 wt. % tocopheryl acetate, and 0.01 wt. % trypan blue (mixed with the tocopheryl acetate by ultrasonic agitation for 15 minutes at 70° F.). The tocopheryl acetate (Vitamin E) is created using an ester of Acetic Acid and Tocopherol being used as an alternative to pure Tocopherol (or undiluted Vitamin E). Tocopheryl acetate has photo-protective properties which can help protect skin against ultraviolet radiation. FIG. 5E confirms that the tagged tocopheryl acetate component of the hydrating serum has transited and taken residence in the stratum corneum, epidermis and dermis.

Serum 4: Serum 4 is composed of 97 wt. % hydration solution (described above), 2.99 wt. % ascorbyl palmitate, and 0.01 wt. % trypan blue (mixed with the tocopheryl acetate by ultrasonic agitation for 15 minutes at 70° F.). Ascorbyl palmitate (Vitamin C) is reacted using an ester combination of ascorbic acid and palmitic acid to form a fatty acid vitamin C to enhance its solubility in both fat and water. A major role of vitamin C is in manufacturing collagen. Ascorbyl palmitate is also an effective free radical-scavenging antioxidant. It also acts synergistically with vitamin E, helping to regenerate the vitamin E radical on a constant basis. FIG. 5F confirms that the ascorbyl palmitate has transited the stratum corneum in significant quantities and has taken residence in the epidermal and dermal layers of skin.

Serum 5: Serum 5 is composed of 95 wt. % hydration solution (described above), 4.99 wt. % niacinimide, and 0.01 wt. % trypan blue (mixed with the tocopheryl acetate by ultrasonic agitation for 15 minutes at 70° F.). Niacinimide, (Vitamin B3) has been described as improving skin's elasticity, dramatically enhance its barrier function, help erase discolorations, and revive skin's healthy tone and texture. It has also been shown to increase ceramide and free fatty acid levels in skin, prevent skin from losing water content, and stimulate microcirculation in the dermis. FIG. 5G clearly shows that the niacinimide has transited the stratum corneum and significant quantities have infiltrated the epidermis and dermal layers of skin tissue.

### Example 13

### Skin Permeation Results

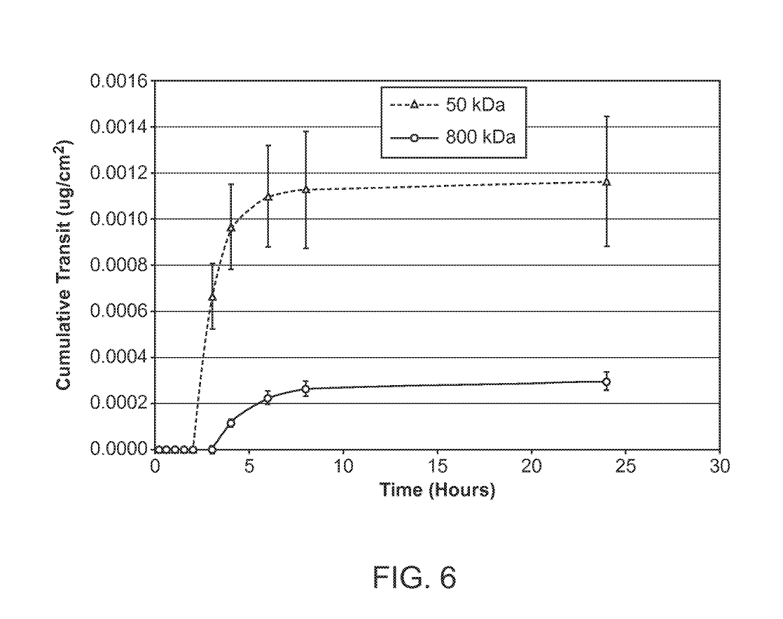

A common tissue donor was used with five independent site applications of the masks tested on tissue specimens of 500 μm thickness through an incubation period of 24 hours with various sampling points in between. Both, the 50 kDa and the 800 kDa masks showed transit into the skin specimens at this incubation point (FIG. 6). Also noted from the results was that the 50 kDa samples showed greater transit into the skin when compared with the 800 kDa samples. Also note that there is no visible initial permeation for the first few hours, but that does not account for the amount of HA retained still within the skin graft prior to diffusion into the receptor chamber.

Also of note here is that the data shown in FIG. 6 denotes the permeation of the HA through a 500 μm over a duration of 24 hours. It does not account for the amount of active material retained within the skin specimen. Following the 24 hour incubation duration, the samples were processed for retention (see methods for details) and results for retention as well as overall uptake (retention +permeation) are shown in Table 1. Also of note is that the 50 kDa samples produced greater transit when compared with the 800 kDa samples by a factor of greater than 2×. It can be speculated that the lower molecular weight HA may provide for an immediate effect onset while the larger molecular weight HA will have a slower release sustained mechanism of action. All results presented are normalized as detailed in the methods section.

Finally, it is visible from these results that the amount of HA retained within the skin graft, superficially, is magnitudes greater than that permeated past the skin graft and into the receptor chamber of the Franz cell setup. This indicates that under real time in vivo conditions, the HA is expected to stay localized to the area under contact with the mask rather than diffuse away laterally or longitudinally. The implication of this is that the active ingredients will remain localized to the anatomical area of interest and focal application.

FIG. 6 is a plot showing transit of the HA into the skin grafts that were 500 μm in thickness. Note greater transit in the 50 kDa samples, while still significantly visible transit with the 800 kDa samples. Table 1 shows quantified data of transit of the HA into the skin grafts that were 500 μm in thickness. Note approximately 3× greater transit in the 50 kDa samples, while still significantly visible transit with the 800 kDa samples. Note that the numbers shown above are reflective of the skin graft after removal of the stratum corneum via tape stripping. It explains why the numbers are a few magnitudes lower than those seen in the stratum corneum alone.

| Transit | 50 kDa | 800 kDa |
|---|---|---|
| Permeation (μg) | 0.0009 ± 0.0002 | 0.0002 ± 0.0000 |
| Retention (μg) | 237.83 ± 41.83 | 105.47 ± 10.09 |
| Uptake (μg/cm²) | 302.81 ± 53.26 | 134.28 ± 12.85 |

These results indicate that the unique excipient being used creates penetration into the stratum corneum and then interacts with structured lipids in the intercellular channels and releases them, thereby enhancing the penetration of hydrophilic actives through the channels. Additionally, the penetration enhancer penetrates into the intracellular matrix of the corneum that fluidizes the intracellular lipids and causes the reduction of diffusional resistance.

Without being bound by theory, we interpret our data to indicate that a combination of both, the lower and higher molecular weight offers a sustained release mechanism and effect. The hyaluronidases family of enzymes potentially has more of a rapid and short term effect on the smaller molecular weight HA than the higher molecular weight. Also, the higher molecular weight HA will continue to absorb water over a longer period of time, thereby providing a dual effect of short and long term sustained release.

### Example 14

### Clinical Results

A study was performed to evaluate the effect of the compositions disclosed herein as adjunctive agents in Ablative or Fractional Ablative Laser Resurfacing.

Facial resurfacing procedures are usually performed in a dermatologic surgeon's office and, depending on technique and area of treatment, can last between 30 minutes to 2 hours. After ablative or fractional ablative laser resurfacing, an ointment is usually applied to facilitate healing, and the patient's face may be covered with a bandage for several days. Significant drainage may occur from the ablated area for up to 2 weeks. Laser-ablated skin can take up to 4 weeks to heal completely and may remain pink to red (erythematous) for several months. Novel adjunctive measures that facilitate healing are critical to improve patient satisfaction and quality of life.

The primary objective of this study is to determine the effectiveness of the Polylaser dry mask and the Polyhydrate mask in reducing healing time, redness, edema, and dried exudate when applied after an ablative or fractional ablative laser resurfacing procedure.

A secondary objective of this study is to measure patient satisfaction with reduction in healing time, redness, edema, and amount of dried exudate after post-laser resurfacing (ablative or fractional ablative) application of Polylaser dry mask followed by the Polyhydrate mask. For the studies disclosed herein, the hydration solutions disclosed herein were used in the masks.

The study was designed as a multicenter, open-label study designed to evaluate the effectiveness of Polylaser dry mask followed by the Polyhydrate mask. After ablative or fractional ablative laser resurfacing, one Polylaser dry mask was applied and kept in place for 24 hours. Three Polyhydrate masks were applied at home over the next three days, once a day for at least 1 hour.

The study population comprises subjects who will receive ablative or fractional ablative laser resurfacing. Subjects who meet all of the following criteria were included in the study:
1. Females age 18 years or older (92.4% of population undergoing non surgical skin rejuvenation procedures are female)
2. Subjects who are already scheduled for ablative laser resurfacing
3. Able and willing to provide written acknowledgment of participation
4. Able to apply mask reliably, as recommended by the provider, either by self or with available assistance
5. Able and willing to maintain patient log for reporting results

Subjects who meet any of the following criteria were excluded from the study:
1. Have received surgical or nonsurgical cosmetic procedures (including facials) at any time during the 4 weeks prior to initiation of the study
2. Are scheduled to receive surgical or nonsurgical cosmetic procedures at any time over the duration of study (30 days)
3. Are pregnant, lactating, or planning to become pregnant
4. Have an open or healing lesion, rash, or other irritation on the face
5. Have or have had a skin disorder that may confound measurement of effectiveness variables or render subject susceptible to complications from ablative or abrasive resurfacing procedures (e.g., skin cancer, scleroderma, dermatitis)
6. Have severe active facial acne
7. Are unable or unwilling to avoid excessive sun exposure or the application of topical products that contain glycolic acid, alpha hydroxyl acids, or retinoids. (Over the course of study, must be willing to apply sunscreen daily.)

A total of 15 subjects were enrolled at three sites.

The following study procedure was employed.

In the screening process, a demographic assessment, medical history assessment, review of compatibility with inclusion and exclusion criteria, and review of informed consent will be performed. Patients who met eligibility standards were enrolled, assigned a subject identification number, and scheduled a first study visit.

On Day 0, the provider will take high-resolution photographs of the subject's face before treatment. The photographs will include a full front view, a 45-degree angle view, and one side view for each side. The provider will then perform an ablative or fractional ablative laser resurfacing procedure and make the following post-treatment assessments:
High-resolution photographs of the face and treated areas (full front view, 45-degree view, and side views)
Area of wound
Redness
Edema
Dried exudate

The provider applied one Polylaser dry mask to the face. Subjects were instructed to keep the mask in place for 24 hours. Subjects were given three Polyhydrate masks and instructed to apply one mask per day, for at least an hour, over the next three days.

On Days 1, 7, and 30, the provider made the following assessments:
High-resolution photographs of the face and treated areas (full front view, 45-degree view, and side views)
Area of healing
Redness
Edema
Dried exudate
Qualitative provider assessment of healing (log entry)
Patient-reported satisfaction (log entry)

The criteria for evaluation included:
Area of healing—Percentage of the total treatment area that had healed
Redness—Scaled provider assessment
Edema—Scaled provider assessment
Dried exudate—Scaled provider assessment
Qualitative provider assessment—Providers kept a log to document the extent of healing, redness, edema, and dried exudate and willingness to use the product with further treatments
Patient-reported satisfaction—Patients kept a log to document their satisfaction with the healing process and willingness to use product with treatments

At each study visit, for each subject, data were collected on case report forms and stored according to HIPAA guidelines. Photographs were evaluated during the course of the study and reviewed only by clinical staff in the practice and the sponsor of the study. All photographs were stored as de-identified data.

Data was collected using patient and physician/aesthetician surveys. Patient and physician preference was determined based on responses to provided surveys.

For physician (i.e., provider) surveys, the following questions were posed.

Providers were asked to rate their level of agreement (strongly disagree, disagree, neutral, agree, or strongly disagree) with the below statements, among others, at various days post-treatment:
I am impressed with the patient's overall recovery.
The healing is taking place more quickly compared to my standard post-treatment protocol.

Providers were also asked to rate the degree (none present, slightly present, some present, present, strongly present) which the below characteristics were present in their patients at various days post-treatment.
Redness
Swelling
Pain
Itchiness

For patient surveys, the following questions were posed.

Patients were asked to rate their level of agreement (strongly disagree, disagree, neutral, agree, or strongly disagree) with the below statements, among others, at various days post-treatment:
I am happy with the way my face is healing.
My face appears less red today.
My face appears less swollen today.
I am feeling some pain from my treatment today.
My face feels less itchy today.
I have had a treatment like this before, and my healing was improved using this product.

### Results

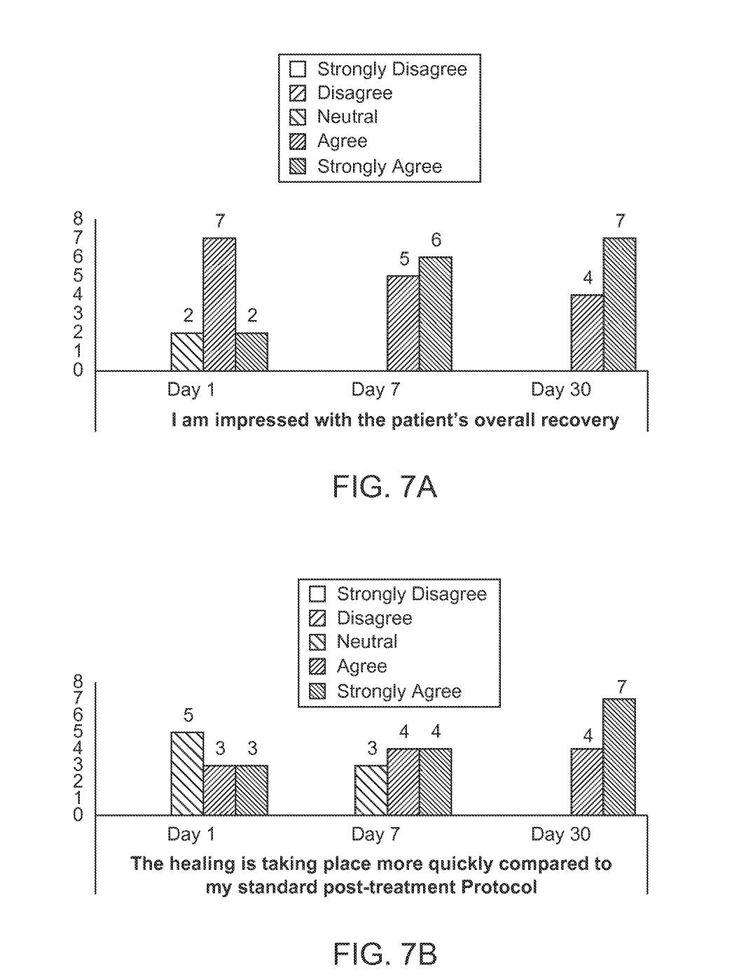

Examples of provider feedback are shown in FIGS. 7-9.

FIG. 7A shows that by Days 7 and 30, the providers agreed or strongly agreed that they were impressed by the patients' recovery in all of the subjects. Furthermore, as shown in FIG. 7B, by Day 30, the providers agreed or strongly agreed that recovery was improved over the standard of care.

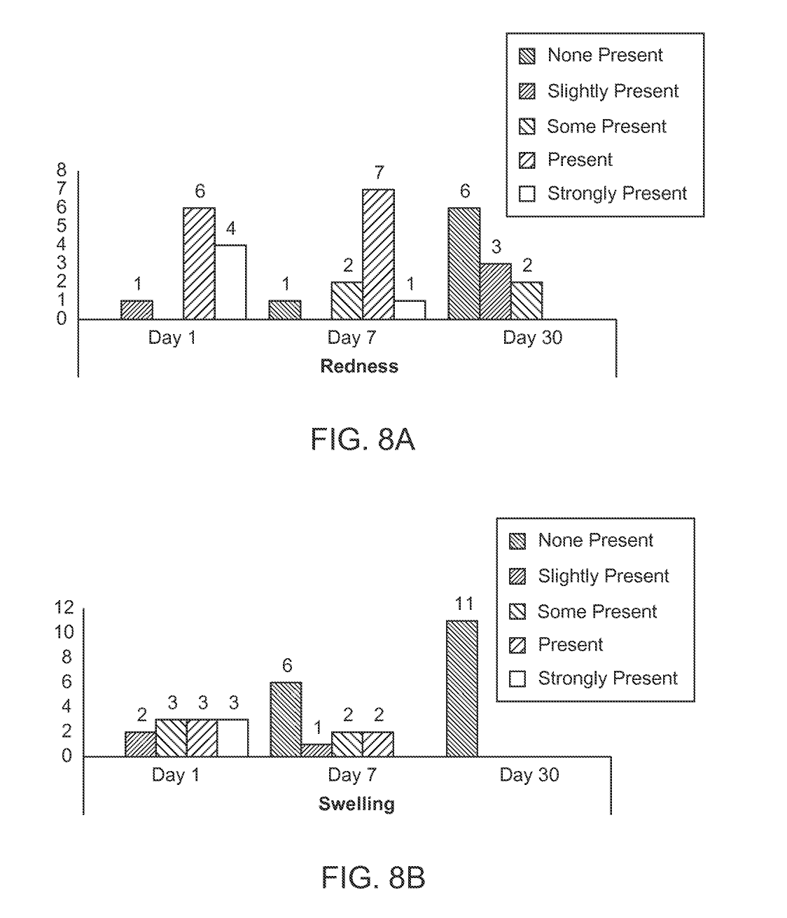

FIG. 8A shows that treatment with serum 5 resulted in decreased amounts of redness throughout the trial. FIG. 8B shows that the amount of swelling also decreased throughout the trial.

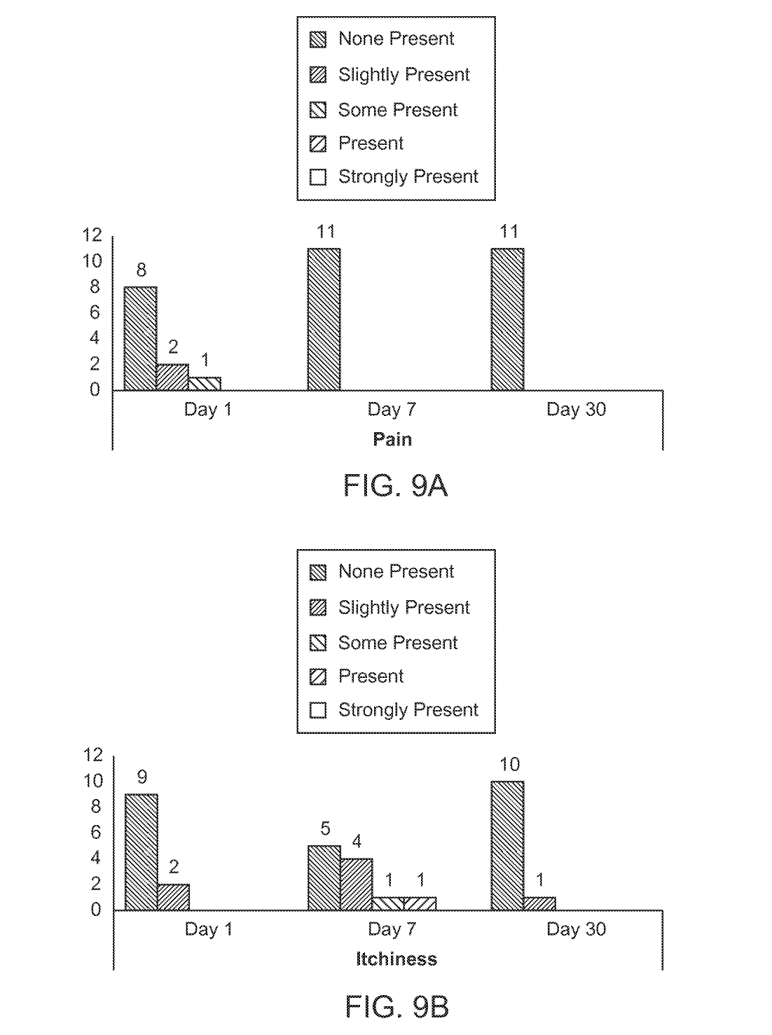

FIG. 9A shows that the amount of pain decreased during the trial, and FIG. 9B shows that the amount of itchiness also decreased during the trial.

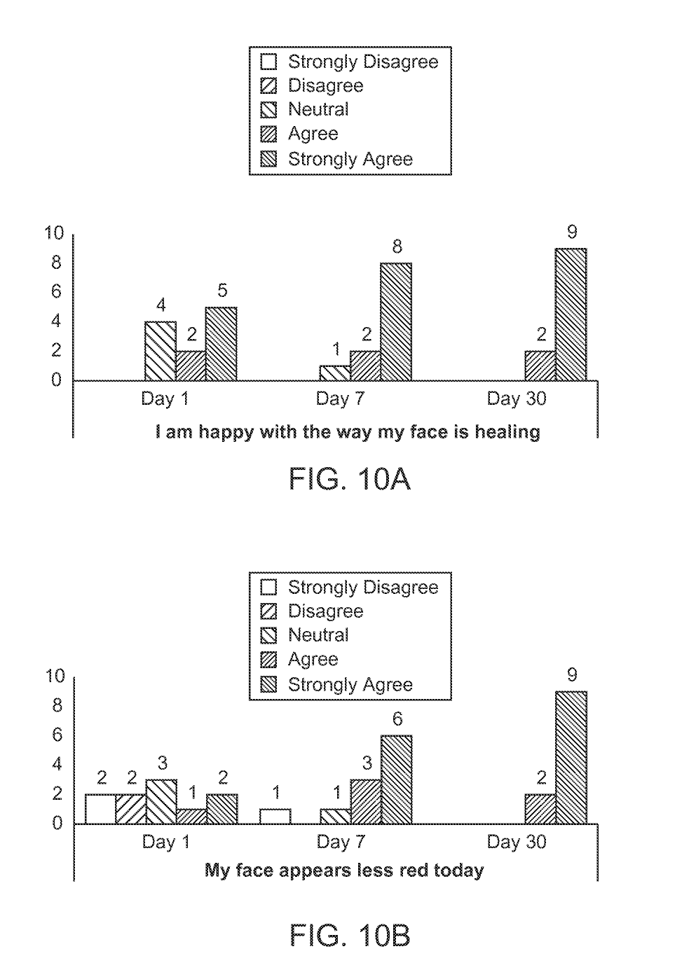

Examples of patient feedback are shown in FIGS. 10-12.

FIG. 10A shows that by Day 30, all the patients in the study were happy with the way their face was healing. Moreover, the patients also experienced a decrease in the amount of erythema as reflected in face redness throughout the trial as shown in FIG. 10B.

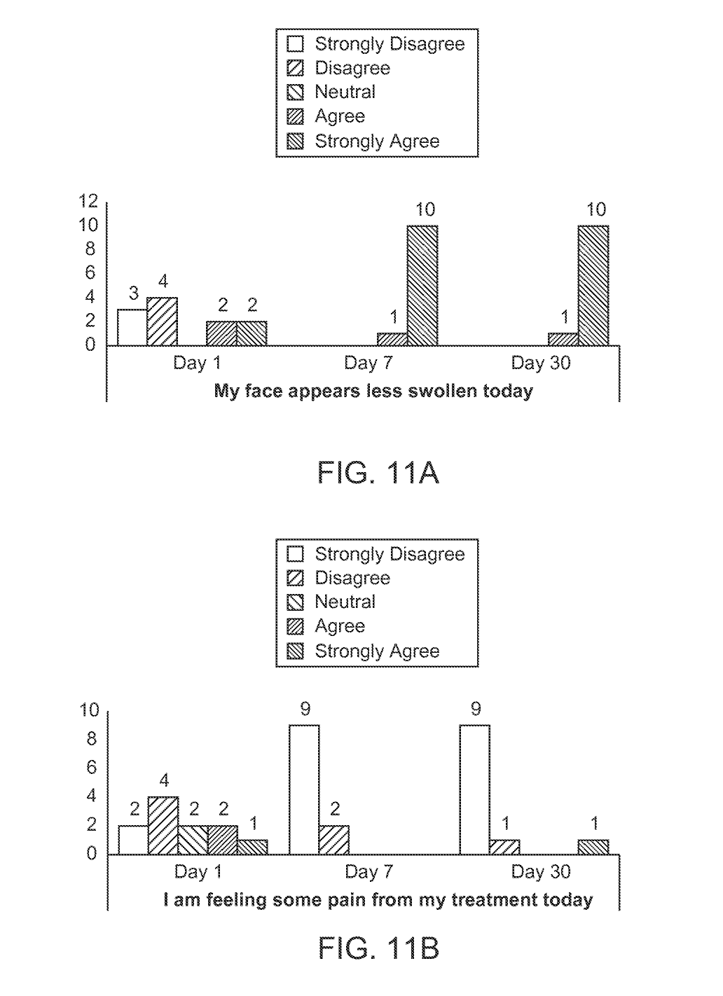

FIG. 11A shows that the patients experienced a decreased amount of swelling throughout the trial, and FIG. 11B shows that the patients experienced a decreased amount of pain throughout the trial.

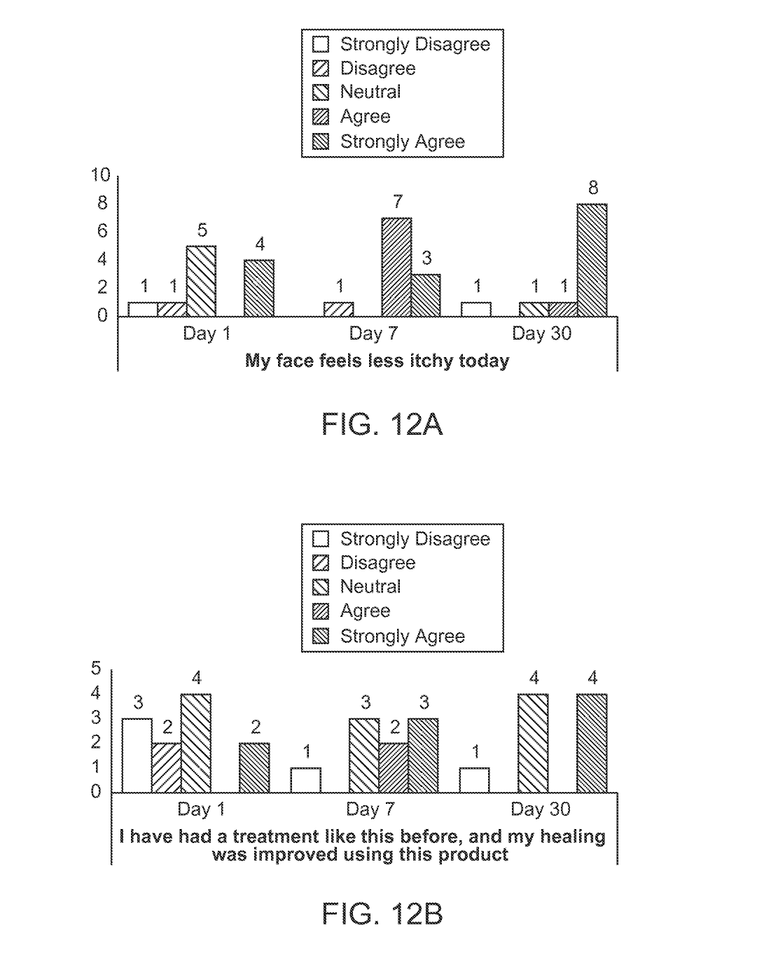

FIG. 12A shows that the patients experienced a decreased amount of itchiness throughout the trial. FIG. 12B shows that at Day 30, 4 out of 9 patients strongly agreed that their healing was improved as compared to their previous treatment, as compared to 2 out of 11 patients on Day 1.

### Example 15

### Effects on Wrinkle Reduction

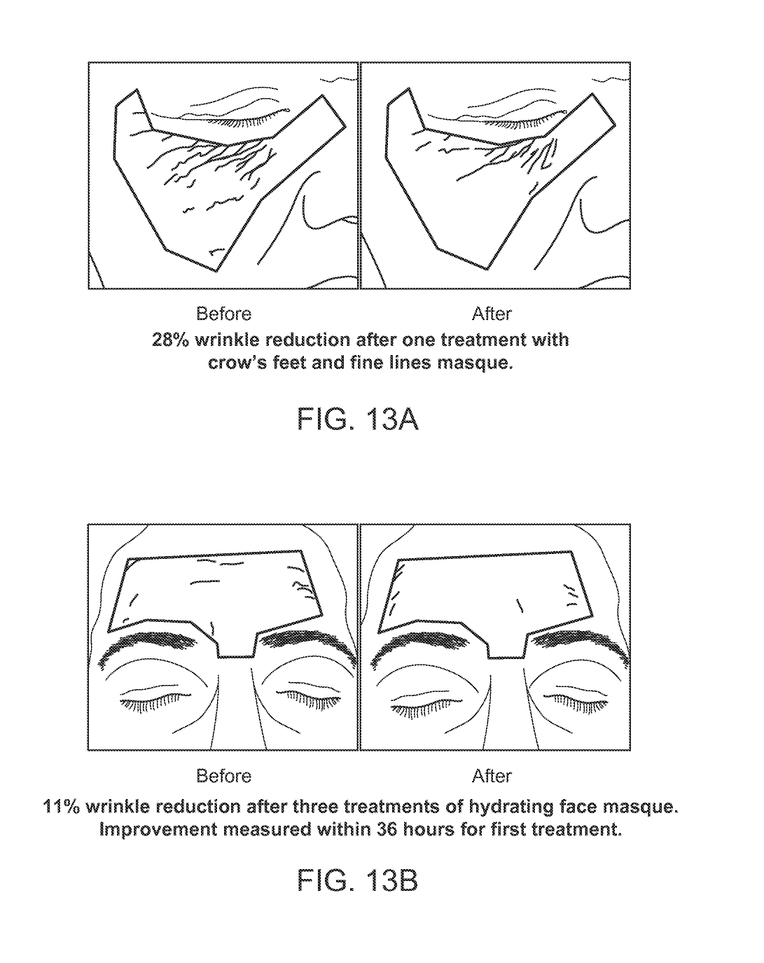

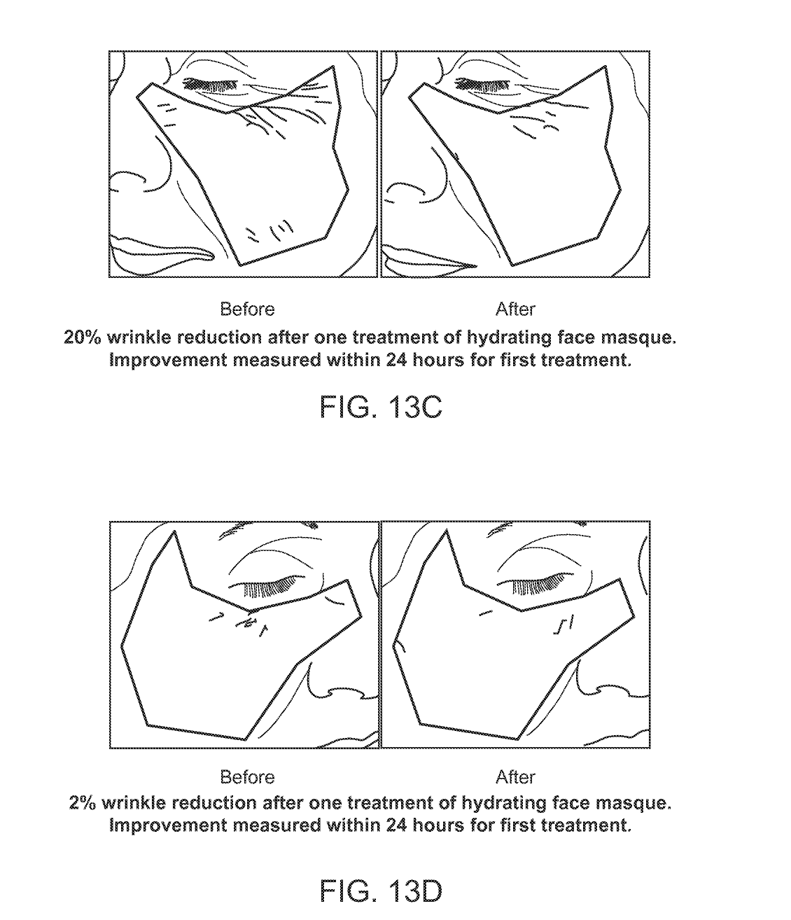

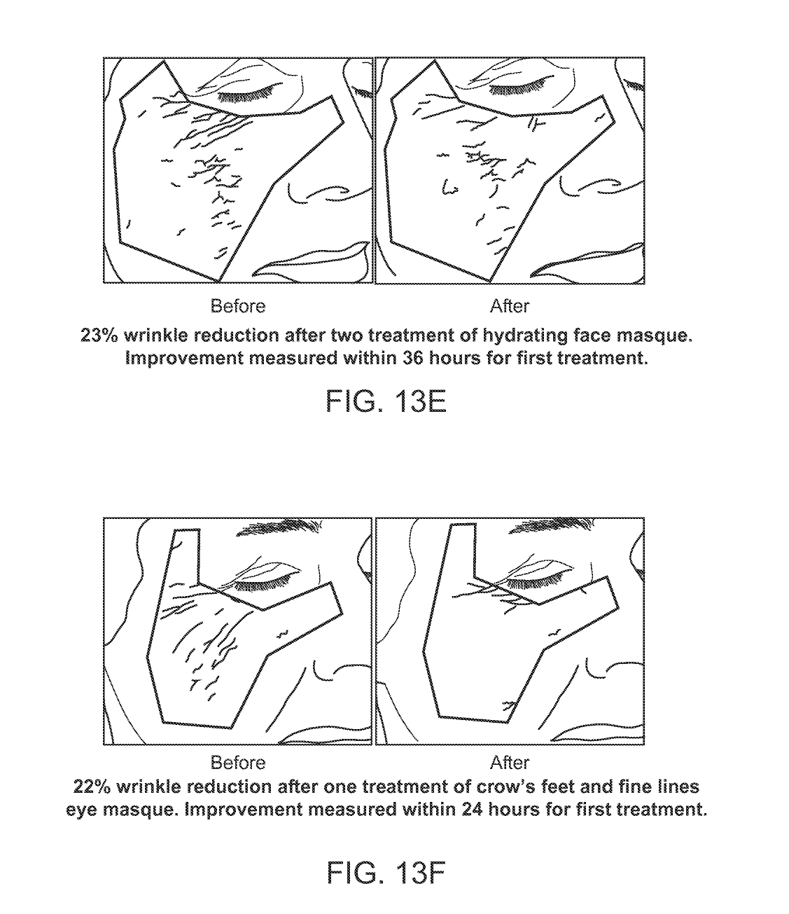

The hydration solutions disclosed herein were also used in studies to determine their effect on wrinkle reduction. Hydration solution was applied to the faces of subjects with hydrating face masques. After varying periods of treatment, the effect of the treatment was determined by measuring wrinkle reduction as shown in FIG. 13.

### Further Considerations

The foregoing description of the embodiments of the disclosure has been presented for the purpose of illustration; it is not intended to be exhaustive or to limit the claims to the precise forms disclosed. Persons skilled in the relevant art can appreciate that many modifications and variations are possible in light of the disclosure herein.

The figures depict various embodiments of the present disclosure for purposes of illustration only. One skilled in the art will readily recognize from the discussion herein that alternative embodiments of the structures and methods illustrated herein may be employed without departing from the principles described herein.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Many modifications and other embodiments of the inventions set forth herein will easily come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

As used in this specification and the appended claims, the singular forms “a,” “an” and “the” include plural references unless the content clearly dictates otherwise.

The use of the alternative (e.g., “or”) should be understood to mean either one, both, or any combination thereof of the alternatives.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

“Comprise,” “comprising,” and “comprises” and “comprised of as used herein are synonymous with “include,” “including,” “includes,” or “contain,” “containing,” “contains” and are inclusive or open-ended terms that specifies the presence of what follows e.g. component and do not exclude or preclude the presence of additional, non-recited components, features, element, members, steps, known in the art or disclosed therein.

As used herein, the terms “such as,” “for example” and the like are intended to refer to exemplary embodiments and not to limit the scope of the present disclosure.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, preferred materials and methods are described herein.

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior invention or for any other reason.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the following claims.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the invention, which is set forth in the following claims.

1. A topical composition, comprising:
5-25% poloxamer;
2-50% isopropyl alcohol (IPA); and
0-30% therapeutic or cosmetic component.

2. The topical composition of claim 1 further comprising 1-50% of a penetration enhancer.

3. The topical composition of claim 1 or 2 comprising 4% or 4.5% poloxamer.

4. The topical composition of claim 1, 2, or 3, wherein the poloxamer is poloxamer 188.

5. The topical composition of any one of claims 1-4 comprising 4.7% laurocapram.

6. The topical composition of any one of claims 1-4, wherein the penetration enhancer is octyl salicylate.

7. The topical composition of any one of claims 1-4, wherein the penetration enhancer is N,N-dialkyl-substituted amino acetate.

8. The topical composition of any one of claims 1-7, wherein the therapeutic or cosmetic component comprises 2-6% hyaluronic acid.

9. The topical composition of claim 8, wherein the therapeutic or cosmetic component comprises 1.6% hyaluronic acid.

10. The topical composition of claim 8, wherein the therapeutic component is 1-3% 50 kDa hyaluronic acid and 1-3% 800 kDa hyaluronic acid.

11. The topical composition of any one of claims 1-10 further comprising 1-25% salicylic acid.

12. The topical composition of claim 11 comprising about 4% salicylic acid.

13. The composition of claim 1, further comprising a component selected from the group consisting of: methanol, ethanol, other water-soluble alcohols, alkyl methyl sulfoxides, dimethyl acetamide, dimethyl formamide, pyrrolidones, propylene glycol, glycerol, silicone fluids, isopropyl palmitate, anionic surfactants, dioctyl sulphosuccinate, sodium lauryl sulphate, decodecylmethyl sulfoxide), bile salts, sodium ms taurocholate, sodium deoxycholate, sodium tauroglycocholate, propylene glycol-oleic acid, 1,4 butane diol-linoleic acid, urea, N,N-dimethyl-toluamide, calcium thioglycolate, anticholinergic agents, eucalyptol, di-o-methyl-beta cyclodextrin, and soyabean.

14. A method of hydrating skin, the method comprising topically applying to skin any one of the compositions of claim 8 or 9.

15. A method of treating rosacea or acne, the method comprising topically applying to skin any one of the compositions of claims 8-12.

16. A method of delivering an active agent through the stratum corneum, the method comprising topically applying to skin any one of the compositions of any one of claims 1-12.

17. A topical composition comprising 95 wt. % hydration solution and 4.99 wt. % Argireline NP.

18. A topical composition comprising 98 wt. % hydration solution and 1.99 wt. % beta glucan.

19. A topical composition comprising 97 wt. % hydration solution and 2.99 wt. % tocopheryl acetate.

20. A topical composition comprising 97 wt. % hydration solution and 2.99 wt. % ascorbyl palmitate.

21. A topical composition comprising 95 wt. % hydration solution and 4.99 wt. % niacinimide.

22. A topical composition of any of one of claim 1-12 or 17-21 for use in the treatment of any of the conditions disclosed herein.

23. A method of preparing a micelle concentrate comprising the components of Table 1 by performing the steps disclosed herein.

24. A method of preparing a hydration comprising the components of Table 2 by performing the steps disclosed herein.

25. A method of preparing a bio-cellulose hydration fluid comprising the components of Table 3 by performing the steps disclosed herein.

26. A method of preparing a formulation for treating acne and/or rosacea comprising the components of Table 4 by performing the steps disclosed herein.
